(19) United States
(12) Patent Application Publication
(10) Pub. No.: US 2006/0127455 A1
(43) Pub. Date: Jun. 15, 2006

(54) Optimized food for expressing luminescent proteins in animals

(75) Inventors: Alan Blake, Austin, TX (US); Richard Crockett, New Haven, CT (US)

Correspondence Address: Vista IP Law Group LLP, 2040 MAIN STREET, 9TH FLOOR, IRVINE, CA, 92614, US

(73) Assignee: Yorktown Technologies, Inc.

(21) Appl. No.: 11/011,822

(22) Filed: Dec. 14, 2004

(51) Int. Cl. A01K 67/027; A23K 1/165; A23K 1/17

(52) U.S. Cl. 424/442; 800/20; 426/635

(57) ABSTRACT

Animal food comprises material which improves and/or optimizes the expression of luminescent proteins in animals. The animals may naturally express luminescent proteins or they may be transgenic animals transfected with a non-endogenous luminescent gene. The animal has a predetermined proteome and the luminescent protein has a predetermined composition of amino acids. The animal food has a base food that is fortified with one or more amino acids which are over-represented in the luminescent protein compared to the amino acids present in the animal proteome. The animal food may further comprise caloric material which increases the cellular metabolism of the animal thereby increasing the expression of the luminescent protein. The animal food may also include inducing material which induces expression of an inducible luminescent gene present in the animal. The animal food may comprise amino acids which are present in, or improve production of, at least one animal tissue in which the luminescent protein is present. The animal food may also comprise a carotinoid and/or a luminescent material.

### FIELD OF THE INVENTION

This invention relates generally to food for animals which luminesce, and more particularly to food which is formulated to increase and/or improve the luminescence of the animal.

### BACKGROUND OF THE INVENTION

There are many species of animals which naturally produce luminescent proteins such that the animals emit visible light in the presence of the appropriate conditions. Luminescence is a phenomenon in which energy is specifically channeled to a molecule to produce an excited state. Return to a lower energy state is accompanied by release of a photon (hy). There are several different types of luminescence, including fluorescence, phosphorescence, chemiluminescence and bioluminescence. The different forms of luminescence are characterized by the type of process which produces the excited state. For example, for fluorescence, the higher energy state is caused by the absorption of excitation light. Accordingly, fluorescence refers to the emission of light by a molecule by a process in which the molecule is excited by the absorption of a photon of a specific wavelength (or range of wavelengths) and upon return to a lower energy state emits a photon of a higher wavelength (and lower energy than the absorbed photon).

Luminescent proteins have many practical uses in pharmaceutical and biotechnology research. For instance, the genes expressing these luminescent proteins have been cloned and exploited as reporter genes in numerous assays, for a wide variety of purposes. As an example, fluorescent proteins can be used to add a fluorescent label to a protein. Such labeling has many valuable uses, including monitoring gene expression and signal transduction pathways, tracking proteins or subcellular organelles, or simply labeling whole cells.

Bioluminescence is the process by which living organisms emit light in which the creation of the excited state derives from an enzyme catalyzed reaction. The color of the emitted light in a bioluminescent (or chemiluminescent or other luminescent, for that matter) reaction is characteristic of the excited molecule, and is independent of its source of excitation and temperature.

An essential condition for bioluminescence is the use of molecular oxygen, either bound or free in the presence of a luciferase (an enzyme protein). Luciferases are oxygenases that act on a substrate, luciferin (another protein or formulated substrate). In the presence of molecular oxygen, luciferase causes a reaction to occur between the oxygen and the luciferin which transforms the substrate to an excited state. When the substrate returns to a lower energy state, energy is released in the form of light [for further background, see, e.g., McElroy et al. (1966) in Molecular Architecture in Cell Physiology, Hayashi et al., eds., Prentice-Hall, Inc., Englewood Cliffs, N.J., pp. 63-80; Ward et al., Chapter 7 in Chemi- and Bioluminescence, Burr, ed., Marcel Dekker, Inc. NY, pp. 321-358; Hastings, J. W. in (1995) Cell Physiology:Source Book, N. Sperelakis (ed.), Academic Press, pp 665-681; Luminescence, Narcosis and Life in the Deep Sea, Johnson, Vantage Press, NY, see, esp. pp. 50-56].

Though rare overall, bioluminescence is more common in marine organisms than in terrestrial organisms. Bioluminescence has developed from as many as thirty evolutionarily distinct origins and, thus, is manifested in a variety of ways so that the biochemical and physiological mechanisms responsible for bioluminescence in different organisms are distinct. Bioluminescent species span many genera and include microscopic organisms, such as bacteria [primarily marine bacteria including Vibrio species], fungi, algae and dinoflagellates, to marine organisms, including arthropods, mollusks, echinoderms, and chordates, and terrestrial organism including annelid worms and insects.

There are also a number of organisms which produce fluorescent proteins causing these organisms to fluoresce in the presence of the proper excitation light. Some of the more commonly known organisms are the jellyfish Aequorea Victoria and reef coral in the class Anthozoa. The jellyfish Aequorea Victoria produces what is generally known as green fluorescent protein (“GFP”). Because of its usefulness in pharmaceutical and biotechnology research, GFP is well characterized and more information can be obtained in the numerous publications describing GFP and its uses (see for example, U.S. Pat. No. 5,491,084; U.S. Pat. No. 5,625,048; U.S. Pat. No. 6,066,476; and U.S. Pat. No. 6,090,919). Substantial research has been conducted with the Aequorea GFP resulting in several mutations of the gene to modify the expression, brightness and emission and excitation spectra. Several of these modified GFPs, including enhanced GFP (eGFP), are described in detail in U.S. Pat. No. 5,625,048, U.S. Pat. No. 6,066,476, U.S. Pat. No. 6,090,919 and U.S. Pat. No. 6,172,188. Numerous types of GFP are commercially available from Amersham Biosciences.

The reef coral fluorescent proteins (“RCFP”) have been derived from a variety of specific Anthozoa species, including Anemonia majano, Discosoma striata, Discosoma sp. “red,” Discosoma sp “green.” Many of the RCFPs are discussed in detail in the published PCT Application Serial No. PCT/US00/28477. The RCFPs and mutations thereof are described in detail in published PCT Application No. PCT/US00/28477. The RCFPs are commercially available from BD Biosciences Clontech.

Luminescent proteins have many practical uses in pharmaceutical and biotechnology research. Because of their usefulness in research, many luminescent proteins have been isolated, studied, modified, optimized and characterized. For instance, the genes expressing these luminescent proteins have been cloned and exploited as reporter genes in numerous assays, for a wide variety of purposes. As an example, fluorescent proteins can be used to add a fluorescent label to a protein. Such labeling has many valuable uses, including monitoring gene expression and signal transduction pathways, tracking proteins or subcellular organelles, or simply labeling whole cells.

More specifically, luciferase genes have been cloned and exploited as reporter genes in a wide variety of assays. Since the different luciferase systems have different specific requirements, they may be used to detect and quantify a variety of substances. The majority of commercial bioluminescence applications are based on firefly [Photinus pyralis] luciferase. One of the first and still widely used assays involves the use of firefly luciferase to detect the presence of ATP. It is also used to detect and quantify other substrates or co-factors in the reaction. Any reaction that produces or utilizes NAD(H), NADP(H) or long chain aldehyde, either directly or indirectly, can be coupled to the light-emitting reaction of bacterial luciferase.

Another luciferase system that has been used commercially for analytical purposes is the Aequorin system. The purified jellyfish photoprotein, aequorin, is used to detect and quantify intracellular Ca.sup. 2+ and its changes under various experimental conditions. The Aequorin photoprotein is relatively small [about 20 kDa], nontoxic, and can be injected into cells in quantities adequate to detect calcium over a large concentration range [3.times.10.sup.−7 to 10.sup.−4 M]. Many of these luminescent luciferases and substrates [e.g., firefly luciferase is available from Sigma, St. Louis, Mo., and Boehringer Mannheim Biochemicals, Indianapolis, Ind.; recombinantly produced firefly luciferase and other reagents based on this gene or for use with this protein are available from Promega Corporation, Madison, Wis.; the aequorin photoprotein luciferase from jellyfish and luciferase from Renilla are commercially available from Sealite Sciences, Bogart, Ga.; coelenterazine, the naturally-occurring substrate for these luciferases, is available from Molecular Probes, Eugene, Oreg.]. These luciferases and related reagents are used as reagents for diagnostics, quality control, environmental testing and other such analyses.

Luminescent proteins are especially useful for in vivo experiments and assays because the proteins are generally non-toxic and can be positioned under control of specific promoters. Accordingly, researchers have created numerous transgenic animals transfected with luminescent proteins for conducting various experiments in vivo, including developmental and gene expression analysis, carcinogenic testing, and other pharmacological studies. Some of the more common animals used for creating transgenic animal models are fish, mouse, rat and hamster.

PCT Application Serial No. PCT/SG99/0079, International Publication No. WO 00/49150, by Gong et al., discloses many different types of transgenic fluorescent fish and various methods of producing such fish. For instance, the zebrafish, Danio Rerio, transfected with eGFP is described in detail. Zebrafish are an excellent experimental model because of several distinct advantages such as the easy availability of eggs and embryos, tissue clarity throughout embryongenesis, short generation time and low maintenance of adult and young zebrafish. In addition, numerous modified mutants of GFP are disclosed, for example, various colors and mammalian optimized mutants are described. As discussed above, fluorescence is the emission of light resulting from the absorption of excitation light. For example, GFP has a maximum excitation at a wavelength of 395 nm and emits green fluorescence at a wavelength (maximum) of 508 nm. The transgenic ornamental fish described in PCT/SG99/0079 are genetically engineered by introducing genes into the fish which express fluorescent proteins. By positioning the fluorescent gene under the control of a specific promoter, the fluorescent protein genes may be used to express the fluorescent proteins in specific tissues, such as in skin tissue, muscle tissue or bone tissue. Gong et al. disclose fish containing numerous different fluorescent proteins, including green fluorescent protein (GFP), enhanced green fluorescent protein (eGFP), yellow fluorescent protein (YFP), enhanced yellow fluorescent protein (eYFP), blue fluorescent protein (BFP), enhanced blue fluorescent protein (eBFP), cyan fluorescent protein (CFP) and enhanced cyan fluorescent protein (eCFP).

Currently, these transgenic luminescent animals are typically fed their ordinary feed for that type of animal. However, such feed does not necessarily comprise the nutritional content which will improve or optimize the expression of the luminescent proteins in the animals. It would be advantageous to formulate animal feed which is optimized for expressing the luminescent proteins in the animals.

All patents, patent applications and other publications referenced in this application are hereby incorporated by reference herein in their entirety.

### SUMMARY OF THE INVENTION

The animal food of the present invention is specifically formulated to improve and/or optimize the luminescence of transgenic luminescent animals. More specifically, the animal food comprises enhanced nutritional content, such as amino acids, that are over-represented in the luminescent proteins with respect to the known proteome of the animal. In addition, the food may include increased caloric content as the production of luminescent protein may require additional cellular metabolism. In many cases, the luminescent protein is expressed in a specific tissue of the animal such as in bone tissue, muscle tissue or skin tissue. In such case, the animal food may comprise amino acids or other material which increase the production of the specific tissue in which the luminescent protein is expressed.

By performing a frequency analysis of the amino acids comprising the luminescent protein and the proteome of the animal, the animal food can be fortified with the amino acids utilized more often in the cellular production of the luminescent protein.

In addition to fortification with amino acids, the fish food may also contain “carotenoids”, such as beta-carotene and lycopene, which are known to increase fish color. In a further aspect of the present invention, the animal food may also include a luminescent material added to the food. For example, the luminescent material may comprise fluorescent materials, bio-luminescent material, phosphorescent materials, fluorescent proteins, or chemiluminescent materials.

In addition, the animal food may be specially formulated to improve the brightness of colored pigments in transgenic animals which may not be luminescent. The animal food may contain amino acids, proteins or other materials which improve or enhance the brightness of colored pigments in the transgenic animal.

### DETAILED DESCRIPTION OF THE INVENTION

Embodiments of the invention will now be described. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

The animal food of the present invention may comprise chemical compounds, amino acids and caloric content which are specifically formulated to improve and/or optimize the luminescence of transgenic luminescent animals. The following description is specific to transgenic ornamental fish which express GFP or RCFP, but the invention is not limited to fish. Indeed, the invention encompasses food for any animal which naturally expresses or is transgenically modified to express luminescent proteins. More specifically, the animal food comprises enhanced nutritional content, such as amino acids, that are over-represented in the luminescent proteins with respect to the known proteome of the animal. In addition, the food may include increased caloric content as the production of luminescent protein may require additional cellular metabolism.

A tremendous amount of research has been conducted on transgenically modifying the zebra fish, Danio Rerio, to express fluorescent genes. This work includes the development of numerous gene constructs with varying gene promoters and fluorescent genes designed which to produce the desired gene expression and fluorescent characteristics. PCT/SG99/0079 describes the use of many different types of gene promoters including heterologous and homologous reporters. The gene promoters can be used to determine where, when and under what conditions the fluorescent gene expresses. By applying these different promoters, various expression properties may be obtained, including, tissue-specific expression, constitutive or inducible expression or ubiquitous expression. The isolation of myriad zebrafish gene promoters is described in PCT/SG99/0079, including eye-specific, bone-specific, tail-specific, muscle-specific, hormone inducible, heavy-metal inducible, heat-shock inducible and the like.

Accordingly, the transgenic fish have been produced comprising a fluorescent gene under the control of these promoters which are tissue-specific fluorescent, such as skin fluorescent or muscle fluorescent, or even one color for one tissue type and another color for a different tissue type. The transgenic fish may also be constitutively fluorescent, inducibly fluorescent or ubiquitously fluorescent.

In order to improve or optimize the fluorescence of these transgenic fish, the animal food of the present invention is specifically formulated to comprise enhanced nutritional content, such as amino acids, that are over-represented in the luminescent proteins with respect to the known proteome of the animal. In addition, the food may include increased caloric content as the production of luminescent protein may require additional cellular metabolism. In order to determine how to improve or optimize the animal food, a frequency analysis of the amino acids comprising the luminescent protein and the proteome of the animal is performed. The animal food is then fortified with the amino acids utilized more often in the cellular production of the luminescent protein. The following examples are representative of the present invention but the invention is by no means limited to these examples.

Fluorescent transgenic zebrafish have been developed utilizing the fluorescent proteins eGFP and DsRed. The percentage amino acid make-up of a typical zebrafish is shown in Table 1. The amino acid content of eGFP and DsRed are also shown in Table 1. As Table 1 shows, the amino acids, Aspartic acid, Glutamic acid, Phenylalanine, Histidine, Glycine, Lysine, Methionine, Valine and Tyrosine are over-represented in eGFP compared to the proteome of the zebrafish. In other words, Glycine makes of 9.2% of the protein eGFP and only 6.35% of the proteome of the zebrafish. The comparison for other amino acids can be determined from Table 1. Accordingly, the fish food for improving the fluorescence of the transgenic zebrafish utilizing eGFP is fortified with a combination of one or more of these over-represented amino acids. The fortification may be accomplished by adding meal or natural materials having high levels of these amino acids or by adding the amino acids themselves to a base food.

TABLE 1
Percentage Content of Amino Acids in GFP, DsRed and Zebrafish Proteome

| | | | | | | | Zebrafish |
|---|---|---|---|---|---|---|---|
| | | | | | | | (Danio Rerio) |
| | | | GFP | (%) | DsRed | (%) | (%) |
| A | Ala | Alanine | 7 | 2.9% | 5 | 2.2% | 6.65% |
| B | Asx | Asparagine or aspartic acid | 0 | 0.0% | 0 | 0.0% | 0.00% |
| C | Cys | Cysteine | 2 | 0.8% | 1 | 0.4% | 2.19% |
| D | Asp | Aspartic acid | 18 | 7.6% | 12 | 5.3% | 5.34% |
| E | Glu | Glutamic acid | 15 | 6.3% | 19 | 8.4% | 6.80% |
| F | Phe | Phenylalanine | 12 | 5.0% | 12 | 5.3% | 3.84% |
| G | Gly | Glycine | 22 | 9.2% | 22 | 9.8% | 6.35% |
| H | His | Histidine | 9 | 3.8% | 8 | 3.6% | 2.55% |
| I | Ile | Isoleucine | 12 | 5.0% | 10 | 4.4% | 4.84% |
| K | Lys | Lysine | 20 | 8.4% | 23 | 10.2% | 6.15% |
| L | Leu | Leucine | 18 | 7.6% | 14 | 6.2% | 9.06% |
| M | Met | Methionine | 7 | 2.9% | 7 | 3.1% | 2.58% |
| N | Asn | Asparagine | 14 | 5.9% | 6 | 2.7% | 4.09% |
| P | Pro | Proline | 11 | 4.6% | 12 | 5.3% | 5.38% |
| Q | Gln | Glutamine | 8 | 3.4% | 7 | 3.1% | 4.55% |
| R | Arg | Arginine | 7 | 2.9% | 10 | 4.4% | 5.42% |
| S | Ser | Serine | 11 | 4.6% | 12 | 5.3% | 8.15% |
| T | Thr | Threonine | 15 | 6.3% | 10 | 4.4% | 5.55% |
| V | Val | Valine | 17 | 7.1% | 19 | 8.4% | 6.36% |
| W | Trp | Tryptophan | 1 | 0.4% | 3 | 1.3% | 1.16% |
| Y | Tyr | Tyrosine | 12 | 5.0% | 13 | 5.8% | 3.01% |
| Z | Glx | Glutamine or glutamic acid | 0 | 0.0% | 0 | 0.0% | 0.00% |

A similar analysis and formulation may be performed for the transgenic zebrafish having the fluorescent gene DsRed. Again referring to Table 1, DsRed has a relatively high content of Lysine, Glycine, and Valine compared to the zebrafish proteome. Therefore, the improved fish food is fortified with a combination of one or more of these proteins. It should be understood that the fortifications described herein may include any combination of one or more of the amino-acids that are over-represented in the fluorescent proteins and are not required to include each and every one of such amino acids.

In the case of inducibly expressing luminescent animals, the fish food is formulated to comprise the chemical substance which induces expression of the luminescent gene. For a hormone-inducible promoter, the hormone of interest, or chemical compound which cause the animal to produce the hormone, is added to the animal food. Likewise, for a heavy-metal inducible promoter, the heavy-metal is added to the animal food.

In order to improve or optimize the luminescence of transgenic animals, the fish food may also contain “carotenoids” which are known to increase fish color. Examples of carotenoids are beta-carotene and lycopene, but any other suitable carotenoid may be utilized.

A luminescent material may also be added to the animal food to provide an ornamental feature to the food. The luminescent material may comprise fluorescent materials, bio-luminescent materials, phosphorescent materials, fluorescent proteins, or chemiluminescent materials. For example, aequorin or any of the fluorescent proteins described above are suitable luminescent materials which can easily be added to the optimized fish food such that the food itself will luminesce when subjected to the proper excitation light source. The effect is particularly attractive in the case of an optimized fish food for transgenic luminescent fish because the food is dispensed into the water.

In addition, the animal food may be specially formulated to improve the quality of colored pigments in transgenic animals which may not be luminescent. The animal food may contain amino acids, proteins or other materials which improve or enhance the brightness of colored pigments in the transgenic animal. For example, food for an animal which has been transgenically modified to add or change the color of the animal's pigments may contain material which makes such color brighter, darker or fuller.

While the present invention has been fully described above with particularity and detail in connection with what is presently deemed to be the invention, it will be apparent to those of ordinary skill in the art that many modifications thereof may be made without departing from the principles and concepts set forth herein. Hence, the proper scope of the present invention should be determined only by the broadest interpretation of the appended claims so as to encompass all such modifications and equivalents.

What is claimed is:

1. Animal food for an animal which expresses a luminescent protein having a predetermined composition of amino acids, said animal having a predetermined proteome, the animal food comprising:
a base food material, and
wherein said base food material is fortified with one or more amino acids which are over-represented in said luminescent protein compared to the amino acids of the animal proteome.

2. The animal food of claim 1 wherein said luminescent protein is a fluorescent protein.

3. The animal food of claim 1 wherein said luminescent protein is a bio-luminescent protein.

4. The animal food of claim 1 wherein said luminescent protein is chemiluminescent.

5. The animal food of claim 1 wherein said animal is a fish transfected with a non-endogenous luminescent gene.

6. The animal food of claim 5 wherein said fish is a transgenic fish of the species zebrafish, medaka, goldfish, carp, koi, loach, tilapia, glassfish, catfish, angel fish, discus, eel, tetra, barb, goby, gourami, guppy, Xiphophorus, hatchet fish, Molly fish, or pangasius.

7. The animal food of claim 6 wherein said transgenic fish comprises one or more chimeric luminescent genes encoding one or more fluorescent proteins.

8. The animal food of claim 5 wherein said fish is a stable transgenic fish line obtained by a method comprising the steps of:
(a) obtaining a transgenic fish comprising one or more chimeric fluorescence genes positioned under the control of a promoter, wherein the transgenic fish expresses one or more fluorescent proteins encoded by the one or more fluorescence genes at a level sufficient such that said fish fluoresces upon exposure to an excitation light source; and
(b) breeding the transgenic fish with a second fish to obtain offspring; and
(c) selecting from said offspring a stable transgenic line that expresses one or more fluorescent proteins.

9. The animal food of claim 5 wherein said luminescent gene comprises one or more of the following: GFP, eGFP, BFP, eBFP, YFP, eYFP, CFP, eCFP, RFP, RCFP or DsRed.

10. The animal food of claim 1 wherein said luminescent protein is expressed by an inducible luminescent gene and said animal food comprises an inducing material which induces expression of the inducible luminescent gene.

11. The animal food of claim 10 wherein said material is a hormone.

12. The animal food of claim 10 wherein said inducing material is a heavy-metal.

13. The animal food of claim 1 further comprising caloric material which increases the cellular metabolism of said animal thereby increasing the expression of said luminescent protein.

14. Animal food for an animal which expresses a luminescent protein in at least one predetermined tissue of said animal, said tissue having a predetermined composition of amino acids, said animal having a predetermined proteome, the animal food comprising:
a base food material, and
wherein said base food material is fortified with one or more amino acids which are over-represented in said predetermined tissue compared to the amino acids of the animal proteome.

15. The animal food of claim 14 wherein said luminescent protein is one of a fluorescent protein, a bio-luminescent protein or a chemiluminescent protein.

16. The animal food of claim 14 wherein said animal is a fish transfected with a non-endogenous luminescent gene.

17. The animal food of claim 16 wherein said fish is a stable transgenic fish line obtained by a method comprising the steps of:
(a) obtaining a transgenic fish comprising one or more chimeric fluorescence genes positioned under the control of a promoter, wherein the transgenic fish expresses one or more fluorescent proteins encoded by the one or more fluorescence genes at a level sufficient such that said fish fluoresces upon exposure to an excitation light source; and
(b) breeding the transgenic fish with a second fish to obtain offspring; and
(c) selecting from said offspring a stable transgenic line that expresses one or more fluorescent proteins.

18. The animal food of claim 16 wherein said luminescent gene comprises one or more of the following: GFP, eGFP, BFP, eBFP, YFP, eYFP, CFP, eCFP, RFP, RCFP or DsRed.

19. The animal food of claim 14 wherein said luminescent protein is expressed by an inducible gene expressing said tissue said animal food comprises an inducing material which induces expression of the gene expressing said tissue.

20. The animal food of claim 14 wherein said tissue is one of muscle tissue, skin tissue or bone tissue.
